SEQUENCE LISTING (1 sequence)

SEQ ID NO: 1 - protein, 15 aa, Artificial Sequence
  Description of Artificial Sequence Synthetic GlySer linker
GGGGSGGGGSGGGGS

(19) United States
(12) Patent Application Publication
(10) Pub. No.: US 2007/0020272 A1
(43) Pub. Date: Jan. 25, 2007

(54) Indirectly linked photosensitizer immunoconjugates, processes for the production thereof and methods of use thereof

(76) Inventor: Tayyaba Hasan, Arlington, MA (US)

Correspondence Address: EDWARDS & ANGELL, LLP, P.O. BOX 55874, BOSTON, MA, 02205, US

(21) Appl. No.: 10/554,814

(22) PCT Filed: Apr. 30, 2004
(86) PCT No.: PCT/US04/13430

§ 371 Date: Oct. 28, 2005

Related U.S. Application Data
(60) Provisional application No. 60/466,574, filed on Apr. 30, 2003.

(51) Int. Cl. A61K 39/395; G01N 33/574; C07K 16/46; C07K 16/30

(52) U.S. Cl. 424/155.1; 530/388.8; 530/391.1; 424/178.1; 435/7.23

(57) ABSTRACT

The present invention relates to indirectly linked photosensitizer immunoconjugate (PIC) compositions, methods of preparation and the use of the same in photodynamic therapeutic and diagnostic applications. PICs comprising a photosensitizer indirectly linked to an antibody via a PEGylated polyglutamate chain are described.

### RELATED APPLICATIONS/PATENTS & INCORPORATION BY REFERENCE

This application claims priority to U.S. Ser. No. 60/466,574, filed on Apr. 30, 2003.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“Herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### STATEMENT OF RIGHTS TO INVENTIONS MADE UNDER FEDERALLY SPONSORED RESEARCH

This work was supported by the government, in part, by a grant from the National Institutes of Health, Grant No. ROI AR40352. The government may have certain rights to this invention.

### FIELD OF THE INVENTION

The present invention relates to indirectly linked photosensitizer immunoconjugate compositions for use in photodynamic therapy. Other aspects of the invention are described in or are obvious from the following disclosure (and within the ambit of the invention).

### BACKGROUND OF THE INVENTION

Photodynamic therapy (PDT) is an emerging modality for the treatment of neoplastic and non-neoplastic cellular diseases. Using photodynamic therapeutic approaches, photosensitizers are localized in target tissues, and subsequently activated with an appropriate wavelength of light. Light activation of the photosensitizers generates active molecular species, such as free radicals and singlet oxygen (¹O₂), which can be toxic to target cells and tissues.

In conventional PDT, selectivity is achieved by irradiating only the desired target area/tissue such that the photosensitizer is only activated in that desired target area. Provided that the photosensitizer is non-toxic, only the irradiated areas will be affected, even if the photosensitizer does bind to normal tissues.

Selectivity thus obtained is adequate in certain situations and for certain anatomical sites, such as skin and oral cavity. However, in many situations greater selectivity is necessary, so that colateral damage to non-target tissues can be minimized. Selectivity can be enhanced by attaching photosensitizers to molecular delivery systems that have high affinity for desired target tissue (Hasan, 1992), (Strong et al., 1994). For example, the photosensitizer can be linked to an antibody directed against a cancer-associated antigen to produce a photoimmunoconjugate, or PIC, capable of delivering the photosensitizer directly to tumor cells. The use of PICs offers improved photosensitizer delivery specificity and can thus broaden the applicability of PDT. For example, it has been suggested that PDT might be used effectively in the treatment of small diffuse malignancies present in a cavity, such as the peritoneum or bladder, if the photosensitizer could be made to accumulate with high specificity in malignant cells (Hamblin et al., 1996). This would allow photodynamic destruction of diseased cells while sparing adjacent normal tissues of sensitive organs.

The efficacy of PDT using PICs can be further enhanced by using antibodies that are themselves cytotoxic. For example, many monoclonal antibodies known in the art possess tumoricidal activity. The combined therapeutic use of a cytotoxic and/or tumoricidal antibody and a photosensitizer compound is referred to herein as photodynamic combination therapy or “combination therapy.” Combination therapies advantageously co-localize photosensitizer compounds and cytotoxic/tumoricidal antibodies to the desired target. Combination therapies include therapies where the PIC itself comprises a cytotoxic/tumoricidal antibody, and therapies where a PIC and a separate cytotoxic/tumoricidal antibody are co-administered.

In addition to being useful for therapy, PICs can be used in any situation wherein selective delivery and accumulation of photosensitizers to a target tissue is desirable. This would include the use of PICS as diagnostic tools. For example, PICs comprising tumor specific antibodies and photosensitizers that emit a detectable signal after irradiation, can be used to determine whether or not tumor cells are present in a patient.

Although nearly 20 years has passed since PICs were first conceived (Mew et al., 1983), problems with their design, synthesis, and purification has hindered progress in their clinical application (Hasan, 1982, Sternberg et al., 1998, Yarmush et al., 1993, and Savellano, 2000). PICs often suffer from difficulties in preparation, poor solubility, poor penetration into tissue (Flessner & Dedrick, 1994) and immunogenicity (Maher et al., 1992). The best photosensitizers are typically hydrophobic and lipophilic, whereas antibodies and immunoconjugates must remain water-soluble and disaggregated in order to reach and bind to their designated targets. Furthermore, due to the hydrophobic and highly adsorptive nature of most photosensitizers, it has been very difficult to remove unconjugated, free photosensitizer impurities from PIC preparations. Another problem associated with the use of PICs for therapeutic or diagnostic applications, is the need to use high doses to achieve the desired biological effect. The higher the dose used, the more likely it is that some of the PIC will accumulate in non-target tissues, and thus the higher the risk of damaging non-target tissues or obtaining false positive diagnostic results.

Conjugation of PICs to molecular delivery vehicles may improve their performance in PDT by increasing specificity for and/or uptake by desired target tissues, altering the pharmacokinetics or biodistribution of the PICS, or decreasing phototoxicity to non-target tissue (Hasan, 1992). The physical properties of PICs, such as size, charge, hydrophobicity, and degree of aggregation can be altered by a number of methods known in the art. For example, the conjugation of polyethylene (PEG) to macromolecules by the process of “PEGylation,” is known to extend serum half-life, reduce immunogenicity, increase solubility and reduce aggregation (Delgado, 1992). Macromolecular backbones, such as polylysine, polyglutamate and polyvinyl alcohol, have also been utilized to improve PIC assemblies, allowing for the indirect linkage of a few photosensitizer molecules to a single antibody.

However, none of the previously described PICs that incorporate pegylation and/or indirect linkages via a backbone substantially overcome the aforementioned limitations of PICs. Thus, there is still a need in the art for development of more effective PIC compositions with improved target specificity, reduced non-specific toxicity and/or immunogenicity, high solubility, and minimal contamination with impurities. Furthermore, there is a need for PICs that can be used at significantly lower doses so that the deleterious effects associated with high dose preparations can be mitigated.

### OBJECT AND SUMMARY OF THE INVENTION

The present invention relates to compositions of PICs comprising photosensitizers indirectly linked to antibodies via PEGylated polyglutamate backbones or linkers. The PICs of the present invention have surprisingly beneficial properties that are superior to other PICS known in the art. A unique structural assembly allows PICs of the present invention to achieve loading of about 40 to 50 photosensitizer molecules per antibody molecule, thereby increasing therapeutic efficacy. Loading of the photosensitizer molecules is achieved through indirect linkages comprising a non-toxic PEGylated polyglutamate backbone, which advantageously prevents attachment of the photosensitizer molecules to the antigen recognition site of the antibody, increasing cellular penetration and therapeutic efficacy. As a result of the improved structure described herein for combining photosensitizers with a tumoricidal antibody, PICs of the present invention exhibit unexpected synergistic therapeutic effects in vivo. Thus, PICs of the present invention overcome problems in the art relating to preparation, toxicity, penetration and efficacy.

In one aspect, the present invention provides an indirectly linked PIC composition comprising an antibody, a PEGylated polyglutamate chain, and at least one photosensitizer molecule, wherein a PEGylated polyglutamate chain comprises a linkage between a non-antigen binding region and a photosensitizer molecule. Accordingly, in one embodiment, the present invention relates to a photosensitizer immunoconjugate composition comprising an antibody, a PEGylated polyglutamate chain and at least one photosensitizer molecule, wherein the PEGylated polyglutamate chain is attached to:

a) a non-antigen binding region of the antibody; and

b) at least one photosensitizer molecule

such that the photosensitizer molecule is indirectly linked to the antibody through the PEGylated polyglutamate chain.

In PICs of the present invention, there is no chemical bond between the antibody and the photosensitizer molecule, such that the photosensitizer molecule is indirectly linked to the antibody via the PEGylated polyglutamate chain.

In yet another aspect, the present invention relates to methods of detecting a target cell in a subject. Accordingly, in one embodiment, the present invention relates to a method of detecting a target cell in a subject comprising the steps of:
a) localizing a photosensitizer immunoconjugate composition comprising an antibody indirectly linked to a photosensitizer by a PEGylated polyglutamate chain to the target cell;
b) light activating the composition to illuminate the target cell; and
c) detecting the target cell.

In another yet another aspect, the present invention provides methods for preparing a PIC comprising PEGylating a polyglutamate molecule, conjugating photosensitizer to said PEGylated polyglutamate molecule, and attaching the PEGylated polyglutamate-photosensitizer conjugate to an antibody. Accordingly, in one aspect the present invention relates to methods for the preparation of such PICs comprising the steps of:
a) preparing a PEGylated polyglutamate chain
b) attaching photosensitizer to a PEGylated polyglutamate chain, and
c) attaching a PEGylated polyglutamate chain to a non-antigen binding region of an antibody
   whereby the antibody is indirectly linked to the photosensitizer through the PEGylated polyglutamate chain.

In yet another aspect, the present invention relates to methods of reducing tumor cell growth and/or proliferation in a subject. Accordingly, in one embodiment, the present invention relates to methods of reducing tumor cell growth and/or proliferation in a subject comprising the steps of:
a) providing a therapeutically effective amount of a photosensitizer immunoconjugate composition comprising an antibody indirectly linked to photosensitizer by a PEGylated polyglutamate chain to the tumor cell, wherein the antibody binds with specificity to an epitope present on the surface of a tumor cell;
b) light-activating the composition to produce phototoxic species; and
c) inhibiting the tumor cell growth and/or proliferation.

In yet another embodiment, the present invention relates to a method of reducing tumor cell growth and/or proliferation in a subject comprising the steps of:
a) providing a therapeutically effective amount of a photosensitizer immunoconjugate composition comprising a antibody indirectly linked to photosensitizer by a PEGylated polyglutamate chain to the tumor cell, wherein the antibody binds with specificity to an epitope present on the surface of a tumor cell and exerts an inhibitory effect on growth and/or proliferation of the tumor cell;
b) light-activating the composition to produce phototoxic species; and
c) inhibiting the tumor cell growth and/or proliferation.

In yet another embodiment, the present invention relates to a method of reducing tumor cell growth and/or proliferation in a subject comprising the steps of:
a) providing a therapeutically effective amount of an indirectly linked photosensitizer immunoconjugate composition comprising an antibody indirectly linked to a photosensitizer by a PEGylated polyglutamate chain to the tumor cell, wherein the antibody binds with specificity to a first epitope present on the surface of a tumor cell;
b) providing a therapeutically effective amount of a second antibody to the tumor cell, wherein the antibody binds with specificity to a second epitope present on the surface of a tumor cell and exerts an inhibitory effect on growth and/or proliferation of the tumor cell;
c) light-activating the tumor cell to produce phototoxic species; and
d) inhibiting growth and/or proliferation of the tumor cell.

These and other objects and embodiments are described in or are obvious from and within the scope of the invention, from the following Detailed Description.

### BRIEF DESCRIPTION OF THE DRAWINGS

FIG. 1 shows the efficacy of the indirectly linked PICs of the present invention in reducing ovarian tumor burden in an animal model.

### DETAILED DESCRIPTION

I. Introduction

The present invention provides PIC compositions that overcome many of the problems associated with PICS previously described in the art, and which can be used for various therapeutic and diagnostic applications. PICs of the present invention comprise an antibody, a PEGylated polyglutamate chain, and at least one photosensitizer molecule. The PEGylated polyglutamate chain consists of two types of attachment; the first is to a non-antigen binding region of an antibody, and the second is to one or more photosensitizer molecules. Thus, the PIC compositions of the present invention comprise at least one photosensitizer molecule indirectly linked to an antibody via PEGylated polyglutamate linkers.

In a preferred embodiment, the PEGylated polyglutamate chain is linked to a lysine residue in the non-antigen binding region.

In one embodiment, there are multiple PEGylated polyglutamate chains linked to the non-antigen binding region. In a preferred embodiment there are 2 or 3 PEGylated polyglutamate chains linked to the non-antigen binding region.

Many photosensitizer molecules can be linked to each antibody molecule via these PEGylated polyglutamate chains. For example, up to around 100 photosensistizer molecules can be incorporated into each PIC molecule. However, in a preferred embodiment, each PIC molecule comprises around 20 photosensitizer molecules.

Perferably, PICs of the present invention have low non-specific toxicity, high targeted phototoxicity, optimal antigen binding, high solubility, minimal aggregation, and/or minimal contamination with unconjugated free photosensitizer molecules. Furthermore, the PICs of the present invention typically enable 40 to 50 photosensitizer molecules to be conjugated to a single antibody molecule. This degree of photosensitizer coupling provides a 10 fold excess of photosensitizer in comparison to other PICs (Savellano et al., Photochemistry and Photobiology 77: p 431-439 (2003)), advantageously allowing for lower doses to be used in patients.

The polyglutamate backbone of the present PIC compositions is advantageously associated with reduced toxicity, as compared to, for example, polylysine backbones. Thus, use of a polyglutamate backbone reduces nonspecific toxicity while optimizing targeted phototoxicity. Use of the polyglutamate backbone also minimizes the presence of unconjugated free photosensitizer impurities in the final PIC product. PEGylation of the polyglutamate backbone inhibits aggregation and promotes solubilization of the PIC.

Furthermore, the indirect linkage of the photosensitizer to the hinge region of antibody (as opposed to the antigen recognition site) prevents interference with antigen binding.

The unique structural assembly of PIC compositions of the present invention results in PICs having a combination of highly desirable properties. Thus, the present invention describes a unique method for the preparation of PICs that overcomes problems in the art relating to the preparation, toxicity, penetration and efficacy.

II. Definitions

As used herein the terms “target cell” and “target tissue” refer to those cells or tissues that are the intended target for the binding of a PIC. The target cells and tissues of the present invention can be any cells or tissues that it is desirable to treat or detect using the methods of the present invention, including tumor cells, immune cells, bacterial cells, fungal cells, parasites, or virus infected cells. The term “tumor” as used herein refers to cells, or masses of cells, that are not subject to the normal constraints on cell growth and division, and includes benign tumors and malignant tumors or “cancers.” The term “tumor” as used herein, also encompass cells and tissues that support the survival and/or propagation of a tumor, such as for example, tumor vasculature and stromal cells such as fibroblasts.

As used herein, the term “photosensitizer” means a chemical compound that produces a biological effect upon photoactivation or a biological precursor of a compound that produces a biological effect upon photoactivation.

As used herein the term “antibody” refers to an immunoglobulin molecule, or fragment or portion thereof, that binds to an epitope on an antigen. The term “epitope” refers to any antigenic determinant, and is understood to comprise a region of an antigenic molecule that binds to an antibody or a T cell receptor. Epitopic determinants usually consist of chemically active surface groupings of molecules such as amino acids or sugar side chains and usually have specific three dimensional structural characteristics, as well as specific charge characteristics. Epitopes of the invention can be present, for example, on cell surface receptors.

Preferably, antibodies of the present invention bind with specificity to the desired target cells or tissues. By “bind with specificity” it is meant that the antibody binds to the target cell or tissue, but to non-target cells. Antibodies that bind with specificity to a target cell or tissue (e.g., via recognition of an antigenic determinant thereof) are referred to herein as “tumor-specific.”

As used herein, the term “photosensitizer immunocojugate” or “PIC” refers to a composition in which a photosensitizer is conjugated to an antibody. If the antibody is conjugated to the photosensitizer by a direct chemical bond between the antibody and the photosensitizer, the antibody and the photosensitizer are said to be “directly linked” or “directly conjugated.”

The term “backbone” refers to any chemical moiety incorporated into a PIC that is not an antibody or a photosensitizer moiety. In the PICs of the present invention, the antibody is connected to the photosensitizer via a “linker” backbone. The antibody and the photosensitizer are said to be “indirectly linked” if they are conjugated via such a linker as opposed to by a direct chemical bond between the antibody and the photosensitizer.

As used herein the term “photodynamic therapy” or “PDT” refers to any in vivo application of either photosensitizers or PICs and comprises both therapeutic and diagnostic applications.

The terms “cytotoxic” and “tumoricidal” as used herein, relate to antibodies, or PIC compositions comprising antibodies, that kill or inhibit proliferation of cells, or specifically tumor cells, respectively.

III. Antibodies

The PICS of the present invention comprise antibodies indirectly conjugated to photosensitizers via PEGylated polyglutamate linkers. The purpose of the antibody component of the PICs is to provide specific targeting of the photosensitizer to the desired target cells or tissues.

In a preferred embodiment, the antibody is monoclonal.

Thus, in one embodiment the antibody component of the PIC binds with specificity to an epitope present on the surface of a target cell. Preferably, the antibody binds to a target cell that is a desired target for therapeutic intervention. For example, the target cell can be a tumor cell that is targeted for destruction using photodynamic therapy. Preferably, the target cell comprises a tumor cell.

In yet another embodiment, the antibody binds to a target cell that is a desired target to identify for diagnostic purposes. For example, accumulation of the photosensitizer can be detected to indicate the presence of a tumor, and/or a tumor expressing particular cell surface antigens.

The antibodies of the invention comprise whole native antibodies, bispecific antibodies; chimeric antibodies, fusion polypeptides, polyclonal antibodies, monoclonal antibodies and humanized, monoclonal antibodies. Further, the antibodies of the present invention comprise intact immunoglobulin molecules as well as fragments thereof, such as Fab and Fab′, which are capable of binding the epitopic determinant. In a preferred embodiment, the antibodies of the present invention are monoclonal. In an even more preferred embodiment, the antibodies of the present invention are humanized monoclonal antibodies.

In one embodiment the antibodies of the present invention are any antibodies that bind to epitopes on the surface of any cell. In another embodiment the antibodies of the present invention bind to epitopes on the surface of animal cells. In a further embodiment the animal cells are mammalian cells. In a preferred embodiment the mammalian cells are human cells. In a more preferable embodiment still, the antibodies of the present invention bind to epitopes on the surface of tumor cells.

Examples of antibodies that bind with specificity to tumor cell epitopes include, but are not limited to, IMC-C225, EMD 72000, OvaRex Mab B43.13, 21B2 antibody, anti-human CEA, CC49, anti-ganglioside antibody G(D2) ch14.18, OC-125, F6-734, CO17-1A, ch-Fab-A7, BIWA 1, trastuzumab, rhuMAb VEGF, sc-321, AF349, BAF349, AF743, BAF743, MAB743, AB1875, Anti-Flt-4AB3127, FLT41-A, rituximab, tositumomab, Mib-1, 2C3, BR96, CAMPATH 1H, 2G7, 2A11, Alpha IR-3, ABX-EGF, MDX-447, SR1, Yb5.b8, 17F.11, anti-p75, anti-p64 IL-2R and MLS 102.

Further tumor-specific antibodies known in the art include those described in U.S. Pat. Nos. 6,197,524, 6,191,255, 6,183,971, 6,162,606, 6,160,099, 6,143,873, 6,140,470, 6,139,869, 6,113,897, 6,106,833, 6,042,829, 6,042,828, 6,024,955, 6,020,153, 6,015,680, 5,990,297, 5,990,287, 5,972,628, 5,972,628, 5,959,084, 5,951,985, 5,939,532, 5,939,532, 5,939,277, 5,885,830, 5,874,255, 5,843,708, 5,837,845, 5,830,470, 5,792,616, 5,767,246, 5,747,048, 5,705,341, 5,690,935, 5,688,657, 5,688,505, 5,665,854, 5,656,444, 5,650,300, 5,643,740, 5,635,600, 5,589,573, 5,576,182, 5,552,526, 5,532,159, 5,525,337, 5,521,528, 5,519,120, 5,495,002, 5,474,755, 5,459,043, 5,427,917, 5,348,880, 5,344,919, 5,338,832, 5,298,393, 5,331,093, 5,244,801, and 5,169,774. See also The Monoclonal Antibody IndexVolume 1: Cancer (3^{rd} edition).

Accordingly, the tumor-specific antibodies of the invention can recognize tumors derived from a wide variety of tissue types, including, but not limited to, breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

Epitopes to which tumor-specific antibodies bind are also well known in the art. For example, epitopes bound by the tumor-specific antibodies of the invention include, but are not limited to, those known in the art to be present on CA-125, gangliosides G(D2), G(M2) and G(D3), CD20, CD52, CD33, Ep-CAM, CEA, bombesin-like peptides, PSA, HER2/neu, epidermal growth factor receptor, erbB2, erbB3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R and CO17-1A.

The antibodies of this invention can be prepared in several ways. Methods of producing and isolating whole native antibodies, bispecific antibodies, chimeric antibodies, Fab, Fab′, single chain V region fragments (scFv) and fusion polypeptides are known in the art. See, for example, Harlow and Lane (1988) Antibodies: A Laboratory Manual, Cold Spring Harbor Laboratory, New York (Harlow and Lane, 1988).

Antibodies are most conveniently obtained from hybridoma cells engineered to express an antibody. Methods of making hybridomas are well known in the art. The hybridoma cells can be cultured in a suitable medium, and spent medium can be used as an antibody source. Polynucleotides encoding the antibody can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., pristane.

Another method of obtaining antibodies is to immunize suitable host animals with an antigen and to follow standard procedures for polyclonal or monoclonal production. Monoclonal antibodies (Mabs) thus produced can be “humanized” by methods known in the art. Examples of humanized antibodies are provided, for instance, in U.S. Pat. Nos. 5,530,101 and 5,585,089.

“Humanized” antibodies are antibodies in which at least part of the sequence has been altered from its initial form to render it more like human immunoglobulins. For example, in some humanized antibodies the heavy chain and light chain C regions are replaced with human sequence. In another type of humanized antibody the CDR regions comprise amino acid sequences for recognition of antigen of interest, while the variable framework regions have been converted to human sequences. See, for example, EP 0329400. In a third type of humanized antibody, the variable regions are humanized by designing consensus sequences of human and mouse variable regions, and converting residues outside the CDRs that are different between the consensus sequences. The invention encompasses humanized Mabs.

The invention also encompasses hybrid antibodies, in which one pair of heavy and light chains is obtained from a first antibody, while the other pair of heavy and light chains is obtained from a different second antibody. Such hybrids may also be formed using humanized heavy and light chains.

Construction of phage display libraries for expression of antibodies, particularly the Fab or scFv portion of antibodies, is well known in the art (Heitner, 2001). The phage display antibody libraries that express antibodies can be prepared according to the methods described in U.S. Pat. No. 5,223,409 incorporated herein by reference. Procedures of the general methodology can be adapted using the present disclosure to produce antibodies of the present invention. The method for producing a human monoclonal antibody generally involves (1) preparing separate heavy and light chain-encoding gene libraries in cloning vectors using human immunoglobulin genes as a source for the libraries, (2) combining the heavy and light chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected antigen, thereby isolating one or more species of phagemid containing particular heavy and light chain-encoding genes and antibody molecules that immunoreact with the preselected antigen.

Single chain variable region fragments are made by linking light and heavy chain variable regions by using a short linking peptide. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one variable region and the amino terminus of another variable region. Other linker sequences can also be used. All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region, or a portion thereof. Compositions comprising a biphasic scFv could be constructed in which one component is a polypeptide that recognizes an antigen and another component is a different polypeptide that recognizes a different antigen, such as a T cell epitope.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector for the production of scFvs is pcDNA3 (Invitrogen, San Diego, Calif.) in mammalian cells, described above.

Expression conditions should ensure that the scFv assumes functional and, preferably, optimal tertiary structure. Depending on the plasmid used (especially the activity of the promoter) and the host cell, it may be necessary or useful to modulate the rate of production. For instance, use of a weaker promoter, or expression at lower temperatures, may be necessary or useful to optimize production of properly folded scFv in prokaryotic systems; or, it may be preferable to express scFv in eukaryotic cells.

Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

In one embodiment, the antibody component of PIC binds with specificity to a receptor or an epitope of a receptor-binding molecule present on the surface of a tumor cell. Antibodies of this category include, but are not limited to, IMC-C225, EMD 72000, BIWA 1, trastuzumab, rituximab, tositumomab, 2C3, rhuMAb VEGF, sc-321, AF349, BAF349, AF743, BAF743, MAB743, AB1875, Anti-Flt-4AB3127, FLT41-A, CAMPATH 1H, 2G7, alpha IR-3, ABX-EGF, MDX-447, SR1, Yb5.B8, 17F.11, anti-p75 IL-2R and anti-p64 IL-2R. Receptor epitopes or an epitope of a receptor-binding molecule include, but are not limited to those known in the art to be present on CD20, CD52, CD33, HER2/neu, epidermal growth factor receptor, erbB3, erbB4, CD44v6, VEGF, VEGFRs (e.g., VEGFR-3), estrogen receptors, TGFβ1, IGF-1 receptor, EGFa, c-Kit receptor, transferrin receptor, and IL-2R.

In a preferred embodiment, the antibody component of the PIC is IMC-C 225, a chimeric therapeutic antibody made to the extracellular domain of the EGFR, which has shown great success in the treatment of head and neck cancer when administered in combination with radiation (Fan and Mendelsohn., 1998). Autocrine activation of the EGFR by EGF and TGF-α is important to tumor cell proliferation, and the EGFR appears to be an excellent target for anti-cancer therapies given that it is overexpressed in several types of tumors such as ovarian, colon, lung, and oral cancer (Perkins, 1997).

In another preferred embodiment, the antibody component of the PIC is a tumoricidal antibody. Antibodies that possess tumoricidal activity are also known in the art, including IMC-C225, EMD 72000, OvaRex Mab B43.13, anti-ganglioside G(D2) antibody chl4.18, C017-1A, trastuzumab, rhuMAb VEGF, sc-321, AF349, BAF349, AF743, BAF743, MAB743, AB1875, Anti-Flt-4AB3127, FLT41-A, rituximab, 2C3, CAMPATH 1H, 2G7, Alpha IR-3, ABX-EGF, MDX-447, anti-p75 IL-2R, anti-p64 IL-2R, and 2A11.

IV. Photosensitizers

The PICS of the present invention comprise antibodies indirectly conjugated to photosensitizers through PEGylated polyglutamate linkers. The photosensitizer of the present invention can be any photosensitizer wherein, after the PIC has been internalized in a target cell, the photosensitizer is capable of being activated by irradiation with light such that it produces a biological effect, or produces a precursor compound that produces a biological effect. Photosensitizers of the invention can be any known in the art, including the following:

Photosensitizers of the invention can be any known in the art, including the following:

a. Porphyrins and Hydroporphyrins

Porphyrins and hydroporphyrins of the invention include, but are not limited to, Photofrin® RTM (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin (IV) chlorin e₆, meta-tetrahydroxyphenylchlorin, chlorin e₆ monoethylendiamine monamide, verdins such as, but not limited to zinc methylpyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium (III) texaphyrin.

Porphyrins, hydroporphyrins, benzoporphyrins, and derivatives are all related in structure to hematoporphyrin, a molecule that is a biosynthetic precursor of heme, which is the primary constituent of hemoglobin, found in erythrocytes. First-generation and naturally occurring porphyrins are excited at 630 nm and have an overall low fluorescent quantum yield and low efficiency in generating reactive oxygen species. Light at ˜630 μm can only penetrate tissues to a depth of 3 mm, however there are derivatives that have been ‘red-shifted’ to absorb at longer wavelengths, such as the benzoporphyrins BPD-MA (Verteporfin). Thus, these ‘red-shifted’ derivatives show less collateral toxicity compared to first-generatio porphyrins.

Chlorins and bacteriochlorins are also porphyrin derivatives, however these have the unique property of hydrogenated exo-pyrrole double bonds on the porphyrin ring backbone, allowing for absorption at wavelengths greater than 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further red-shifted to ˜740 nmn.

Purpurins, porphycenes, and verdins are also porphyrin derivatives that have efficacies similar to or exceeding hematoporphyrin. Purpurins contain the basic porphyrin macrocycle, but are red-shifted to ˜715 nm. Porphycenes have similar activation wavelengths to hematoporphyrin (˜635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals.

5-aminolevulinic acid (ALA) is a precursor in the heme biosynthetic pathway, and exogenous administration of this compound causes a shift in equilibrium of downstream reactions in the pathway. In other words, the formation of the immediate precursor to heme, protoporphyrin IX, is dependent on the rate of 5-aminolevulinic acid synthesis, governed in a negative-feedback manner by concentration of free heme. Conversion of protoporphyrin IX is slow, and administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of 630 nm, offering no advantage in terms of depth of tissue penetration.

b. Cyanine and Other Photoactive Dyes

Photoactive dyes of the invention include, but are not limited to, merocyanines, phthalocyanines with or without metal substituents, chloroaluminum phthalocyanine with or without varying substituents, sulfonated aluminum PC, ring-substituted cationic PC, sulfonated A1Pc, disulfonated and tetrasulfonated derivative, sulfonated aluminum naphthalocyanines, naphthalocyanines with or without metal substituents and with or without varying substituents, tetracyanoethylene adducts, nile blue, crystal violet, azure β chloride, rose bengal, benzophenothiazinium compounds, phenothiazine derivatives including methylene blue.

Cyanines are deep blue or purple compounds that are similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maximum at about 680 μm. Phthalocyanines, belonging to a new generation of substances for PDT are chelated with a variety of metals, chiefly aluminum and zinc, while these diamagnetic metals enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, with high tumor to tissue ratios (8:1) reached after 1-3 hours. The cyanines are eliminated rapidly and almost no fluorescence can be seen in the tumor after 24 hours.

Other photoactive dyes such as methylene blue and rose bengal, are also used for PDT. Methylene blue is a phenothiazine cationic dye that is exemplified by its ability to specifically target mitochondrial membrane potential. Specific tumoricidal effects in response to cationic phenothiazine dyes are thought to be due to the electrical potential across mitochondrial membranes in tumor cells. Compared to normal cells, the potential in tumor cells is much steeper, leading to a high accumulation of compounds with delocalized positive charges (i.e. cationic photosensitizers). Rose-bengal and fluorescein are xanthene dyes that can be used in PDT. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.

c. Other Photosensitizers

Other photosensitizers of the invention include, but are not limited to, Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, tetracyclines and other photosensitizers that do not fall in either of the aforementioned categories have other uses besides PDT, but are also photoactive. For example, anthracenediones, anthrapyrazoles, aminoanthraquinone compounds are often used as anticancer therapies (i.e. mitoxantrone, doxorubicin). These drugs have reasonable tumor selectivity, however adverse side effects and toxicity are common. Chalcogenapyrylium dyes such as cationic selena- and tellurapyrylium derivatives have also been found to exhibit photoactive properties in the 600-900 nm range, more preferably from 775-850 nm. In addition, antibiotics such as tetracyclines and fluoroquinolone compounds have demonstrated photoactive properties.

First-generation photosensitizers are exemplified by the porphyrin derivative Photofrin®, also known as porfimer sodium. Photofrin® is derived from hematoporphyrin-IX by acid treatment and has been approved by the Food and Drug Administration for use in PDT. Photofrin® is characterized as a complex and inseparable mixture of monomers, dimers, and higher oligomers. There has been substantial effort in the field to develop pure substances that can be used as successful photosensitizers. Thus, in a preferred embodiment, the photosensitizer is a benzoporphyrin derivative (“BPD”), such as BPD-MA, also commercially known as Verteporfin. U.S. Pat. No. 4,883,790 describes BPDs. Verteporfin has been thoroughly characterized (Richter et al., 1987; Aveline et al., 1994; Levy, 1994) and it has been found to be a highly potent photosensitizer for PDT. Verteporfin has been used in PDT treatment of certain types of macular degeneration, and is thought to specifically target sites of new blood vessel growth, or angiogenesis, such as those observed in “wet” macular degeneration. Verteporfin is typically adminstered intravenously, with an optimal incubation time range from 1.5 to 6 hours. Verteporfin absorbs at 690 μm, and is activated with commonly available light sources.

In one embodiment, the photosensitizer is a benzoporphyrin derivative (“BPD”), such as BPD-MA, also commercially known as BPD Verteporfin. U.S. Pat. No. 4,883,790 describes BPDs. BPD is a so-called second-generation compound which lacks the prolonged cutaneous phototoxicity of Photofrin® (Levy, 1994). BPD has been thoroughly characterized (Richter et al., 1987), (Aveline et al., 1994), and it has been found to be a highly potent photosensitizer for PDT.

In one embodiment, a compound, e.g., ALA or ALA esters, which causes the accumulation of a photosensitizer, the formation of a photosensitizer, or is converted to a photosensitizer in the subject's body is a photosensitizer of a PIC. For example, a compound which causes the accumulation of, the formation of, or which is converted to, a porphyrin or a porphyrin precursor, is administered to the subject.

In one embodiment, the photosensitizer has a chemical structure that includes multiple conjugated rings that allow for light absorption and photoactivation, e.g., the photosensitizer can produce singlet oxygen upon absorption of electromagnetic irradiation at the proper energy level and wavelength.

In one embodiment of the invention the photosensitizer is a chlorin. In a further embodiment the photosensitizer is chlorin e₆ or a derivative thereof. In a preferred embodiment the photosensitizer is chlorin e₆ monoethylene diamine salt or “CMA.”

The photosensitizers can comprise a plurality of the same, or even different, photosensitizers, covalently linked to a PEGylated polyglutamate linker and thus indirectly linked to an antibody. Similarly, the photosensitizers can comprise a plurality of different photosensitizers or a “cocktail” of photosensitizers indirectly linked to an antibody.

In one embodiment, the invention relates to a PIC wherein the photosensitizer density on the antibody is sufficient to quench photoactivation while the composition is extracellularly located. In this regard, “sufficient to quench photoactivation” means that the photosensitizer molecules are packed densely enough on the antibody to ensure that dequenching cannot occur until PICs are intracellularly localized. Intracellular localization of the PIC occurs through various routes, including receptor-mediated endocytosis. The PICs are dequenched upon intracellular localization into target cells. Intracellular dequenching of the PIC is mediated through hydrolytic and/or enzymatic processes (e.g. lysosomal degradation) and results in enhanced photoactivation upon administration of light. The PICs are less susceptible to photodynamic activation outside of target cells, and thereby produce less collateral damage by way of background photoactivation in non-target tissues.

In a preferred embodiment, the PIC comprises 20 or more photosensitizer molecules each linked indirectly to a single antibody molecule. In an even more preferred embodiment, the PIC comprises 30 or more photosensitizer molecules each linked indirectly to a single antibody molecule. In a more preferred embodiment still, the PIC comprises 40 or more photosensitizer molecules each linked indirectly to a single antibody molecule.

V. Linkers

The PICs of the present invention comprise antibodies indirectly conjugated to photosensitizers through PEGylated polyglutamate chains. These PEGylated polyglutamate chains can comprise any desired number of glutamate residues. In a preferred embodiment each polyglutamate chain comprises 10 to 600 glutamate residues, corresponding to polyglutamate molecules having molecular weights in the range 2000 to 100,000. The polyglutamate molecules can be PEGylated to any level desired. In a preferred embodiment 2 to 10 PEG molecules are coupled to each polyglutamate molecule. PEG is a routinely used laboratory reagent, and PEG from any suitable source or commercial supplier may be used. For example, a wide variety of PEG derivatives are commercially available from Shearwater Polymers, Huntsville, Ala. Suitable PEG derivatives include a 10 kDa two-branched PEG-NHS ester.

VI. Preparation of Indirectly Linked PICs

The present invention relates to photosensitizers that are indirectly linked to antibodies through PEGylated polyglutamate chains to produce high purity PIC compositions. Accordingly, in one aspect the invention relates to methods for the preparation of such PICs comprising the steps of:
a) preparing a PEGylated polyglutamate chain;
b) attaching photosensitizer to a PEGylated polyglutamate chain; and
c) attaching a PEGylated polyglutamate chain to a non-antigen binding region of an antibody
   whereby the antibody is indirectly linked to the photosensitizer through the PEGylated PGA chain.

PICs that have not been pegylated gradually form large insoluble aggregates during long-term storage in DMSO solutions, and it is not possible to transfer concentrated solutions of unPEGylated PICs from DMSO solutions to purely aqueous solutions without forming large insoluble aggregates. To overcome these solubility problems, it is generally accepted in the art that the PIC should comprise a solubility agent, such as PEG or a two-branched PEG-NHS ester. In the present invention the polyglutamate backbone is advantageously PEGylated to overcome PIC aggregation, maintain PIC solubility and reduce reticulo-endothelial system capture of the PIC.

It would be routine practice for one skilled in the art to PEGylate the polyglutamate backbone of the present invention. Any suitable mechanism known to those skilled in the art can be used to PEGylate the polyglutamate backbone. In one embodiment, the polyglutamate backbone can be PEGylated essentially as described in Example 1. The pegylation reaction conditions and times can be varied so long as the reaction conditions and times remain sufficient to allow PEGylation to reach completion. It is preferred that incomplete PEGylation be avoided.

Preferably, the polyglutamate to PEG molar ratio in the conjugation reaction is approximately 1 to 5 as in Example 1. The degree of attachment of PEG to the polyglutamate backbone, which can be accomplished by the reaction of PEG and PGA is controlled by regulation of the amount of PEG in the reaction mixture that is available for binding.

Once the PEGlyation reaction is complete, the PEGylated polyglutamate chain can be purified by one of many techniques known to those skilled in the art. In one embodiment, the PEGylated polyglutamate chain can be purified essentially as described in Example 1.

Any photosensitizer, or a plurality of photosensitizers, can be attached to the PEGylated polyglutamate chain. Any suitable method of synthesis (i.e., chemical reaction scheme) known to those of skill in the art can be used to attach photosensitizers to the PEGylated polyglutamate chain. Preferably, the photosensitizer is chlorin e₆ monoethylene diamine (disodium salt) or “CMA.” Accordingly, in one embodiment, CMA can be attached to the PEGylated polyglutamate chain essentially as described in Example 1. The photosensitizer-PEG-polyglutamate composition so produced can be purified by any suitable technique known in the art. In one embodiment, the photosensitizer-PEG-polyglutamate conjugate can be purified essentially as described in Example 1.

Prior to attachment of the photosensitizer-PEG-polyglutamate chain to an antibody, it is necessary to “activate” the photosensitizer-PEG-polyglutamate composition. By “activation” is meant the creation of a suitable reactive group on the photosensitizer-PEG-polyglutamate composition, which will enable it to react with and bind to an activated antibody. Any suitable activation method known in the art can be used. In one embodiment, the photosensitizer-PEG-polyglutamate composition can be activated with hydrazine as described in Example 1. Activation of a photosensitizer-PEG-polyglutamate composition with hydrazine can result in the formation of a hydrazide group on the carboxylic acid terminus of a glutamate residue. The activated photosensitizer-PEG-polyglutamate composition can be purified by any suitable technique known in the art. In one embodiment, the activated photosensitizer-PEG-polyglutamate composition can be purified essentially as described in Example 1.

Prior to attachment to the photosensitizer-PEG-polyglutamate compositon, the antibody must also be “activated.” By “activation” is meant the creation of a suitable reactive group on the antibody which will enable it to react with and bind to an activated photosensitizer-PEG-polyglutamate composition. Any suitable method known in the art can be used to activate the antibody. In one embodiment the antibody can be activated essentially as described in Example 1, whereby the hydroxyl groups of the carbohydrates in the hinge region of the antibody are oxidized. The activated antibody can be purified by any suitable technique known to those skilled in the art. In one embodiment, the activated antibody can be purified essentially as described in Example 1.

The activated photosensitizer-PEG-polyglutamate composition can be attached to a non-antigen binding region of the activated antibody by any suitable mechanism known to those of skill in the art. In one embodiment the activated antibody is conjugated to the activated photosensitizer-PEG-polyglutamate composition essentially as described in Example 1, i.e. an amide bond is formed between an oxidized hydroxyl group in the hinge region of the activated antibody, and a hydrazide group of the activated photosensitizer-PEG-polyglutamate composition. Preferably, the photosensitizer-PEG-polyglutamate composition is linked to a lysine residue in the antibody hinge region.

The formation of these covalent amide linkages between the photosensitizer-PEG-polyglutamate composition and the carbohydrate in the antibody hinge region, can be controlled by regulation of the amount of photosensitizer-PEG-polyglutamate composition added to the reaction mixture. The reaction should continue for as long as is necessary to ensure that the reaction of the photosensitizer-PEG-polyglutamate composition with the antibody carbohydrate group has gone to completion. Preferably, the reaction should continue for about 16 hours or more.

After the reaction is complete, the conjugation reaction mixture can be purified using any suitable technique known in the art. In one embodiment, the PIC can be purified essentially as described in Example 1.

Prior to use the PIC preparations can be desalted and concentrated by any suitable means known in the art. In one embodiment the PIC is desalted and concentrated as described in Example 1. PIC preparations in PBS can be stored at about 4° C., remaining stable at least for several months. Similarly, PIC preparations can be stored in approximately 50% DMSO/50% aqueous solution at about 4° C., remaining stable at least for several months.

If deemed necessary, the PIC preparations can be sterile filtered prior to use, using a 0.2 μm filter membrane. To reduce the loss of the PIC resulting from non-specific PIC adsorption to the filter membrane, approximately 1 mg of serum albumin for every approximately 100 μg of conjugate can be added to the PIC preparations prior to sterile filtering.

The PIC purity, photosensitizer concentration and the antibody-photosensitizer ratio can be determined using any suitable mechanism known to those of skill in the art, such as for example SDS PAGE and/or spectrophotometric analysis. With attention to detail and proper handling, it is possible to obtain PIC preparations that contain less than about 5% residual free photosensitizer impurity, or preferably, less than about 1% residual free photosensitizer impurity and which comprise about 40 or more photosensitizer molecules indirectly linked to each antibody molecule.

VII. Use of the Indirectly Linked PICs

The PICs of the invention are useful in a variety of therapeutic and diagnostic in vivo applications.

The indirectly linked PICs can be used in photodynamic therapy to inhibit the growth of, or kill, any target cell, such as for example, a tumor cell. Therapeutic applications center generally on treatment of various disorders by administering an effective amount of the PICs of the invention. The PICs of the present invention bind specifically to particular antigens on the surface of target cells, and therefore they are ideally suited for targeted cell specific photodynamic therapy. According to this aspect of the invention, the antibody component of the PIC functions to deliver photosensitizer to the desired target site. In yet another aspect, the present invention relates to methods of reducing tumor cell growth and/or proliferation in a subject.

Accordingly, in one embodiment, the present invention relates to methods of reducing tumor cell growth and/or proliferation in a subject comprising the steps of:
a) administering a therapeutically effective amount of a photosensitizer immunoconjugate composition comprising an antibody indirectly linked to photosensitizer by a PEGylated polyglutamate chain, wherein the antibody binds with specificity to an epitope present on the surface of a tumor cell;
b) localizing the composition to the tumor cell;
c) light-activating the composition to produce phototoxic species; and
d) inhibiting the tumor cell growth and/or proliferation.

The choice of antibodies used to make the PICs of the present invention depends upon the purpose of delivery and the desired target cells. The delivery to specific target cells, and the activation of the PICs of the present invention at specific target sites, can result in selective killing or inhibition of proliferation of target cells.

In vivo administration of the PICs of the present invention may involve use of any suitable adjuvant including serum or physiological saline, with or without another protein, such as human serum albumin. Dosage of the PICs can readily be determined by one of ordinary skill, and may differ depending upon the nature of the target cell and the specific PIC composition used.

After administration of the PICs of the invention to a subject, and localization of the PICs in the desired target cells, the photosensitizer component of the PIC is activated by a light source and its biological effects are mediated, for example through the production of singlet oxygen.

The specificity of the photochemical reaction can be maintained by selecting the proper wavelength and specific photosensitizer to be used depending on the biologic effect desired. It is possible to attach more than one photosensitizer for delivery to a target site. The photosensitizer can be activated at the target site with lasers or other light sources via optical fibers or any other appropriate method.

Accordingly, an embodiment of the invention relates to a method of reducing target cell growth and/or proliferation comprising the steps of administering a therapeutically effective amount of a PIC composition wherein the antibody component of the PIC binds with specificity to an epitope present on the surface of a target cell, and activating the photosensitizer component of the PIC using a suitable light source, wherein the activated photosensitizer exerts an inhibitory effect on the proliferation of, or kills, the target cell.

In yet another aspect, the invention relates to combination therapy methods of treatment, in which the PICs either comprise a cytotoxic/tumoricidal antibody, or are co-administered with a cytotoxic/tumoricidal antibody.

Accordingly, in one embodiment, the present invention relates to a method of reducing tumor cell growth and/or proliferation in a subject comprising the steps of:
a) administering a therapeutically effective amount of a photosensitizer immunoconjugate composition comprising a antibody indirectly linked to photosensitizer by a PEGylated polyglutamate chain, wherein the antibody binds with specificity to an epitope present on the surface of a tumor cell and exerts an inhibitory effect on growth and/or proliferation of the tumor cell;
b) localizing the composition to the tumor cell;
c) light-activating the composition to produce phototoxic species; and
d) inhibiting the tumor cell growth and/or proliferation.

In yet another embodiment, the present invention relates to a method of reducing tumor cell growth and/or proliferation in a subject comprising the steps of:
a) administering a therapeutically effective amount of an indirectly linked photosensitizer immunoconjugate composition comprising an antibody indirectly linked to a photosensitizer by a PEGylated polyglutamate chain, wherein the antibody binds with specificity to a first epitope present on the surface of a tumor cell;
b) localizing the indirectly linked photosensitizer immunoconjugate composition to the tumor cell;
c) administering a therapeutically effective amount of a second antibody, wherein the antibody binds with specificity to a second epitope present on the surface of a tumor cell and exerts an inhibitory effect on growth and/or proliferation of the tumor cell;
d) localizing the second antibody to the tumor cell;
e) light-activating the tumor cell to produce phototoxic species; and
f) inhibiting growth and/or proliferation of the tumor cell.

Methods of this invention are particularly useful wherein the target cell is a tumor cell and wherein the aim is to treat a neoplastic disease. For example, melanoma, neuroblastoma, glioma, sarcoma, lymphoma, ovarian, prostate, colorectal and small cell lung cancers can be treated by using the PICs of the present invention in photodynamic therapy. The methods comprise administering an amount of a pharmaceutical composition containing PICs to a subject to achieve palliation of an existing tumor mass or prevention of recurrence.

The “subjects” or “patients” of the present invention are vertebrates. Preferably the subjects are a mammalian, more preferably the subjects are human. Mammals include, but are not limited to, humans, farm animals, sport animals, and pets.

A “therapeutically effective amount” is an amount sufficient to effect a beneficial or desired clinical result. A therapeutically effective amount can be administered in one or more doses. In terms of treatment, an effective amount is an amount that is sufficient to palliate, ameliorate, stabilize, reverse or slow the progression of a cancerous disease (e.g. tumors, dysplaysias, leukemias) or otherwise reduce the pathological consequences of the cancer. A therapeutically effective amount can be provided in one or a series of administrations. In terms of an adjuvant, an effective amount is one sufficient to enhance the immune response to the immunogen. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the antibody being administered.

The dosage of the PIC compositions and/or tumoricidal antibody compositions can vary from about 0.01 mg/m² to about 500 mg/m², preferably about 0.1 mg/m² to about 200 mg/m², most preferably about 0.1 mg/m² to about 10 mg/m². Ascertaining dosage ranges is well within the skill of one in the art. For example, in phase three clinical studies, IMC-C225 loading in human patients was between 100-500 mg/m², and maintenance was between 100-250 mg/m² (Waksal, 1999). The dosage of photosensitizer compositions can range from about 0.1 to 10 mg/kg. Methods for administering photosensitizer compositions are known in the art, and are described, for example, in U.S. Pat. Nos. 5,952,329, 5,807,881, 5,798,349, 5,776,966, 5,789,433, 5,736,563, 5,484,803 and by (Sperduto et al., 1991), (Walther et al., 1997). Such dosages may vary, for example, depending on whether multiple administrations are given, tissue type and route of administration, the condition of the individual, the desired objective and other factors known to those of skill in the art. For instance, the concentration of scFv typically need not be as high as that of native antibodies in order to be therapeutically effective. Administrations can be conducted infrequently, or on a regular weekly basis until a desired, measurable parameter is detected, such as diminution of disease symptoms. Administration can then be diminished, such as to a biweekly or monthly basis, as appropriate.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with tumoricidal antibodies. Therapeutic compositions of PICs are often administered by injection or by gradual perfusion.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow release or sustained release forms, whereby a relatively consistent level of the active compound are provided over an extended period.

Another method of administration is intralesionally, for instance by direct injection directly into the tumor. Intralesional administration of various forms of immunotherapy to cancer patients does not cause the toxicity seen with systemic administration of immunologic agents (Fletcher and Goldstein, 1987), (Rabinowich et al., 1987), (Rosenberg et al., 1986), Pizza et al., 1984).

For methods of combination therapy comprising administration of a PIC and a tumoricidal antibody or administration of a photosensitizer and a tumoricidal antibody, the order in which the compositions are administered is interchangeable. Concomitant administration is also envisioned.

Methods of the invention are particularly suitable for use in treating and imaging brain cancer. When the site of delivery is the brain, the therapeutic agent must be capable of being delivered to the brain. The blood-brain barrier limits the uptake of many therapeutic agents into the brain and spinal cord from the general circulation. Molecules which cross the blood-brain barrier use two main mechanisms: free diffusion and facilitated transport. Because of the presence of the blood-brain barrier, attaining beneficial concentrations of a given therapeutic agent in the CNS may require the use of specific drug delivery strategies. Delivery of therapeutic agents to the CNS can be achieved by several methods.

One method relies on neurosurgical techniques. In the case of gravely ill patients, surgical intervention is warranted despite its attendant risks. For instance, therapeutic agents can be delivered by direct physical introduction into the CNS, such as intraventricular, intralesional, or intrathecal injection. Intraventricular injection can be facilitated by an intraventricular catheter, for example, attached to a reservoir, such as an Ommaya reservoir. Methods of introduction are also provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes (Neuwelt and Rapoport, 1984), (Baba et al., 1991), (Gennuso et al., 1993).

Further, it may be desirable to administer the compositions locally to the area in need of treatment; this can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel® provided by Guilford Pharmaceuticals Inc.

Methods of the invention are also particularly suitable for use in primary treatment of intraperitoneal cancers, such as ovarian and colorectal cancers and cancer of the bladder. Other potential uses include those where combination therapies could be combined with surgical debulling, such as pleural mesothelioma or advanced stage ovarian cancer. Currently, advanced ovarian cancer is treated by staging/debulking surgery, followed by chemotherapy, which is usually a combination of Taxol and platinum-based regimen. Rather than chemotherapy, combination therapy could instead be administered. For example, an administration scheme is envisioned whereby a PIC composition is administered either before or after maximal debulking and subsequently light activated following the surgical procedure in order to eliminate residual cancer cells. In addition, administration of a photosensitizer or PIC composition, followed by maximal debulking, administration of a tumoricidal antibody, and subsequent light activation is also envisioned.

The PIC compositions of the present invention can be administered in a pharmaceutically acceptable excipient, such as water, saline, aqueous dextrose, glycerol, or ethanol. The compositions can also contain other medicinal agents, pharmaceutical agents, adjuvants, carriers, and auxiliary substances such as wetting or emulsifying agents, and pH buffering agents.

Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

The PICs of the present invention must be photoactivated to induce their intended biological effect. The photoactivating light can be delivered to the target site from a conventional light source or from a laser. Target tissues are illuminated, usually with red light from a laser. Given that red and/or near infrared light best penetrates mammalian tissues, photosensitizers with strong absorbances in the approximately 600 nm to 900 nm range are optimal for PDT. Delivery can be direct, by transillumination, or by optical fiber.

Optical fibers can be connected to flexible devices such as balloons equiped with light scattering medium. Flexible devices can include, for example, laproscopes, arthroscopes and endoscopes.

Following administration of a PIC composition, it is necessary to wait for the photosensitizer to reach an effective tissue concentration at the target site before photoactivation. The duration of the waiting step will vary, depending on factors such as route of administration, target location, and speed of PIC movement in the body. In addition, where PICs target receptors or receptor binding epitopes, the rate of PIC uptake can vary, depending on the level of receptor expression and/or receptor turnover on the target cells. For example, where there is a high level of receptor expression, the rate of PIC binding and uptake is increased. The waiting period should also take into account the rate at which PICs are degraded and thereby dequenched in the target tissue. Determining a useful range of waiting step duration is within ordinary skill in the art and may be optimized by utilizing fluorescence optical imaging techniques.

Following the waiting step, the photosensitizer and/or PIC composition is activated by photoactivating light applied to the target site. This is accomplished by applying light of a suitable wavelength and intensity, for an effective length of time, specifically to the target site. The suitable wavelength, or range of wavelengths, will depend on the particular photosensitizer(s) used. Wavelength specificity for photoactivation depends on the molecular structure of the photosensitizer. Photoactivation occurs with sub-ablative light doses. Determination of suitable wavelength, light intensity, and duration of illumination is within ordinary skill in the art.

The light for photoactivation can be produced and delivered to the tumor site by any suitable means. For superficial targets or open surgical sites, suitable light sources include broadband conventional light sources, broad arrays of light emitting diodes (LED), and defocussed laser beams.

For non-superficial target sites, including those in intracavitary settings, the photoactivating light can be delivered by optical fiber devices. For example, the light can be delivered by optical fibers threaded through small gauge hypodermic needles. Optical fibers also can be passed through arthroscopes, endoscopes and laproscopes. In addition, light can be transmitted by percutaneous instrumentation using optical fibers or cannulated waveguides.

Photoactivation at non-superficial target sites also can be by transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, i.e. lightboxes or convergent light beams.

For photoactivation, the wavelength of light is matched to the electronic absorption spectrum of the photosensitizer so that photons are absorbed by the photosensitizer and the desired photochemistry can occur. Except in special situations, where the targets being treated are very superficial, the range of activating light is typically between approximately 600 and 900 μm. This is because endogenous molecules, in particular hemoglobin, strongly absorb light below about 600 nm and therefore capture most of the incoming photons (Parrish, 1978). The net effect would be the impairment of penetration of the activating light through the tissue. The reason for the 900 nm upper limit is that energetics at this wavelength may not be sufficient to produce ¹O₂, the activated state of oxygen, which without wishing to necessarily be bound by any one theory, is perhaps critical for successful PDT. In addition, water begins to absorb at wavelengths greater than about 900 nm. While spatial control of illumination provides specificity of tissue destruction, it can also be a limitation of PDT. Target sites must be accessible to light delivery systems, and issues of light dosimetry need to be addressed (Wilson, 1989). In general, the amenability of lasers to fiberoptic coupling makes the task of light delivery to most anatomic sites manageable.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (e.g., 700-800 nm) (Svaasand and Ellingsen, 1983). These values can be altered by altering the biologic interactions and physical characteristics of the photosensitizer. Factors such as self-shielding and photobleaching (self-destruction of the photosensitizer during the PDT) further complicate precise dosimetry. In general, photosensitizers with longer absorbing wavelengths and higher molar absorption coefficients at these wavelengths are more effective photodynamic agents.

PDT dosage depends on various factors, including the amount of the photosensitizer administered, the wavelength of the photoactivating light, the intensity of the photoactivating light, and the duration of illumination by the photoactivating light. Thus, the dose of PDT can be adjusted to a therapeutically effective dose by adjusting one or more of these factors. Such adjustments are within ordinary skill in the art.

In yet another aspect, the invention relates to diagnostic methods utilizing PICs. Accordingly, an embodiment of the invention relates to a method of detecting a target cell in a subject comprising the steps of
a) administering a PIC composition comprising antibody indirectly linked to photosensitizer by a PEGylated polyglutamate chain;
b) localizing the composition to the target cell;
c) light activating the composition to illuminate the target cell; and
d) detecting the target cell.

The photosensitizers component of PICs used in diagnostic applications can be any known in the art. In selecting a photosensitizer for diagnostic purposes, fluorochromic properties of the photosensitizer may be of greater importance than photochemical properties. For use of the PICs of the present invention in diagnostic applications, the same factors as described above for therapeutic applications must be taken into consideration, for example factors regarding, choice of PIC, PIC dosage and PIC administration route. In addition, a suitable means of detecting those cells in which the PIC is activated must be employed. Many such detection or “imaging” techniques are known, and the choice of a suitable imaging technique would be routine for one skilled in the art.

The present invention is additionally described by way of the following illustrative, non-limiting Examples, that provide a better understanding of the present invention and of its many advantages.

### EXAMPLES

### Example 1

### Preparation of an Indirectly Linked PIC

The following steps were performed to indirectly couple the photosensitizer chlorin e₆ monoethylene diamine (disodium salt) or “CMA” to the C225 tumoricidal antibody, thus yielding a PIC of the present invention:

Step 1: Pegylation of Polyglutamic acid (PGA)

A. PGA, PEG and 1-ethyl-3-(3-dimethylaminopropyl) carbodiimide (EDC) were dissolved in 5 milliliters of distilled water at a molar ratio of 1:5:10, respectively, and reacted for 24 hours at room temperature with continuous stirring.
B. After 24 hours of reaction, 20 microliters of triethylamine was added and the reaction was allowed to proceed at room temperature for a further 24 hours.
C. The reaction was tested for completion using the niehydrin test.
D. The completed reaction mixture was concentrated to a volume of approximately 1 milliliter using a vacuum freeze-drying system.
E. The reaction was then purified by column chromatography using a sephadex G50 column and eluting with an acetate buffer (pH 5-5.5).
F. Fractions containing a clean absorbance peak at 200 nanometres were pooled and the volume of the combined pools was reduced to approximately 5 milliliters using a vacuum freeze-drying system. This 5 ml sample comprised PEGylated-PGA (“PP”).
   Step 2: Attachment of the Photosensitizer CMA to the Product of Step 1
A. The PP product of step 1, CMA, EDC and N-hydroxysulfosuccinimide (S—NHS) were dissolved at a 1:10:30:30 molar ratio, respectively, in 10 milliliters of 0.1M MES, with the pH adjusted to approximately 9.0 by addition of 1M NaOH. The mixture was reacted for 24 hours at room temperature.
B. A second round of CMA, EDC and S-NHS (at the same molar ratios as in step 2A) was added directly to the reaction mixture of Step 2A and reacted at room temperature for a further 24 hours.
C. The conjugated CMA-PEG-PGA product of Step 2B was purified by column chromatography using a sephadex G50 column and eluting with an acetate buffer (pH 5.5).
D. Desired fractions (i.e., having long-shifted absorption, indicating that free CMA is minimal) were pooled and dried using a vacuum freeze drying system.
   STEP 3: Activation of the CMA-PEG-PGA Product of Step 2 with Hydrazine
A. The CMA-PEG-PGA product of Step 2 was dissolved in 3.7 milliliters of hydrazine. 50 molar equivalents of EDC and 30 molar equivalents of S—NHS was added to the dissolved CMA-PEG-PGA. The mixture was reacted for approximately 12-16 hours.
B. The reaction mixture of Step 3A was dried using a vacuum freeze drying system and purified by column chromatography using a sephadex G50 column, eluting with an acetate buffer (pH 5.5). The dried sample was solubilized by resuspension in PBS column loading.
C. Desired fractions were pooled, dried using a vacuum freeze drying system and re-dissolved in 5 milliliters of distilled water. The CMA concentration was determined by measuring absorbance at 655 nanometers, subtracting the 800 nanometer absorbance, and assuming the extinction coefficient was the same as for free CMA (i.e. 25,250 M⁻¹cm⁻¹).
   Step 4: Activation of the Antibody C225 for CMA-PEG-PGA Conjugation
A. The C225 antibody was concentrated to 5-10 mg/ml by vacuum freeze drying and mixed with 20 mM NaIO4—at a 1:1 volumetric ratio, and incubated for 1 hour at room temperature.
B. Excess oxidation was then quenched by addition of 250 microliters of ethylene glycol per 1 milliliter of reaction volume and incubation at room temperature for 15 minutes.
C. The activated antibody product of Step 4B was purified using sephadex G50 spin columns and eluted in an acetate buffer (pH 5.5).
   Step 5: Conjugation of Activated C225 and activated CMA-PEG-PGA
A. The activated CMA-PEG-PGA of Step 3 and the activated monoclonal antibody C225 of Step 4C were mixed at a molar ration of 1:100 and incubated overnight at 4° C.
B. The conjugated C225-CMA-PEG-PGA product of Step 5A, were purified by standard protein-A chromatography techniques.
C. The eluted fractions were desalted and concentrated using a 100,000 MW cut-off centricon filter.
D. The CMA concentration and antibody-CMA ratio were determined using spectrophotometry.

### Example 2

### Use of an Indirectly Linked PIC to Inhibit Tumor Growth In Vivo

The antibody component of a PIC can possess tumoricidal properties that are independent of the photosensitizer compound to which the antibody is linked. The monoclonal antibody IMC-C225 (C225) possesses tumoricidal properties. Thus, the use of a PIC comprising C225 in photodynamic therapy comprises a “combination therapy.”

PICs of the present invention comprising the tumoricidal antibody C225 indirectly linked to the photosensitizer CMA (referred to here as “C225-CMA”), were evaluated for efficacy in PDT using a xenograft animal model of intra-peritoneal epithelial ovarian carcinoma. Results obtained in this model system are reasonably predictive of treatment efficacy for the human condition. The following groups were analyzed:

Group 1: No Treatment; Group 2: Treatment with indirectly linked C225-CMA and activation with high a high light dose (high fluence rate); Group 3: Treatment with indirectly linked C225-CMA and activation with a low light dose (low fluence rate).

Materials and Methods

To test the effect of the PICs of the present invention in vivo, a known animal model system was utilized (Molpus et al., 1996a). This xenograft model of intra-peritoneal epithelial ovarian carcinoma has been noted to be desirable for measurement of the effects of PDT (Molpus et al., 1996a). This model manifests tumor derived from human ovarian carcinoma cells with all of the inherent biological properties of human disease. As has been previously described, the model is characterized, as in human patients, by diffuse solid tumor, ascites, parenchymal invasion, lymph-vascular space invasion, and neovascularization (Molpus et al., 1996a). Briefly, athymic Swiss female nude mice, weighing 20-25 grams (6-8 weeks old) were injected intraperitoneally, using a 27-gauge needle, with 31.5×10⁶ NIH:OVCAR-5 cells, suspended in 2 ml PBS. NIH:OVCAR-5 cells were obtained from the Fox Chase Cancer Institute (Philadelphia, Pa.). Cells were grown in RPMI-1640 media (Mediatech Inc, Herndon, VA) supplemented with 10% heat-inactivated fetal calf serum (GIBCO Life Technologies, Grand Island, N.Y.), and 100 U/ml penicillin and 100 μg/ml streptomycin. The cells were maintained in an incubator at 37° C. in an atmosphere of 5% CO₂. At the time of NIH:OVCAR-5 cell injection, mice were given a numeric ear tag. Animals were anesthetized before the cell injection with 0.03 ml of a ketamine/xylazine mixture (ketamine, 120 mg/kg; xylazine, 15 mg/kg).

The mice were maintained in accordance with the guidelines established by the Massachusetts General Hospital Subcommittee on Research Animal Care. They had continual access to food and water, taken ad libitum. Animals were housed in laminar flow racks, under specific pathogen-free conditions. Sacrifices were performed by CO₂ inhalation.

Intraperitoneal (“i.p.”) PDT in the nude mice was performed as previously described (Molpus et al., 1996a). On day 10 and 20 after tumor cell injection, mice in treatment groups 2 and 3 were injected with 1 mg/kg body weight of the above PICs and irradiated with a total of 20J of 665 nm light 24 hours later.

Animals which had to be illuminated were injected with 2 mL of a 0.1% intralipid solution i.p. prior to illumination to enhance light scattering. Animals were anesthetized with 0.03 ml of a ketamine/xylazine mixture (ketamine, 35 mg/ml; xylazine, 5 mg/ml). A solid state diode laser was used for illumination (BWF 690-1, B&W TEK, Newark, DE), which delivers monochromatic light (690+/−5 nm) to overlap closely the absorption maximum of CMA (690 nm), at a maximum power from the diode of 1 W. Alternatively, an argon-pumped dye laser (Coherent) was used to deliver 690 nm light i.p. via a cylindrically diffusing fiberoptic tip (8.0 mm×0.4 mm). The fiber, connected to the Argon-pumped dye laser or the solid state diode laser was introduced into the peritoneal cavity of a supine anesthetized animal via a centrally placed 22-gauge catheter traversing the abdominal cavity. A total of 20 J of light was delivered, at a fluence rate of 100-200 mw/cm². Of the total light energy, one fourth (5 J) was delivered to each i.p. quadrant over equivalent time periods. At the completion of the treatment, the mice were allowed to recover in an animal warmer until they awoke and resumed normal activity.

The endpoint studied was short-term tumor weight. Animals were sacrificed on day 21 to assess acute treatment effects. Animals were also carefully examined for the presence of distinct extra-abdominal metastasis. Representative tissue samples were examined pathologically via hematoxylin and eosin staining. Animals were also weighed before tumor cell injection, and before sacrifice at day 21.

Tumoricidal response was assessed by comparing the extent of gross residual disease in treated animals to the extent of disease in untreated controls. Using the distribution pattern of the tumor in the OVCAR-5 human xenograft mouse model, which was previously described (Molpus et al., 1996b), the sites where tumor was consistently present were dissected.

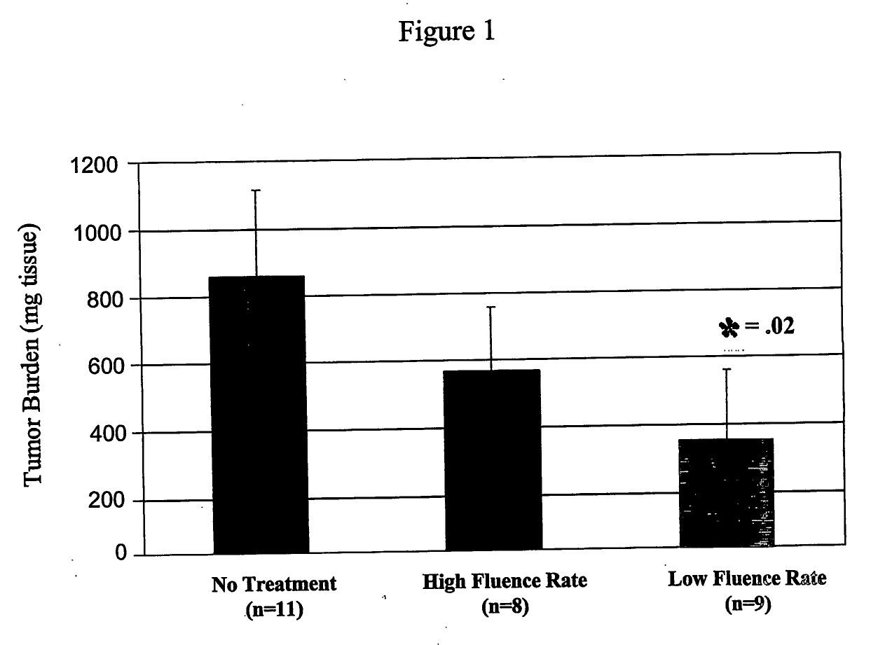

From the results depicted in FIG. 1, it can be seen that administration of indirectly linked C225-CMA with photoactivation at a high fluence rate (Group 2) resulted in a reduced tumor burden as compared to the no treatment group (Group 1). Furthermore, administration of indirectly linked C225-CMA with photoactivation at a low fluence rate (Group 3) resulted in a significantly reduced tumor burden as compared to the no treatment group (p<0.02).

Summary

In humans, recurrent ovarian carcinoma is rarely curable. Prospects for improving survival rates rest on early detection and development of more effective treatment modalities. In advanced stages, ovarian cancer is most frequently limited to the peritoneal cavity. The results presented herein show that anti-cancer treatments directed to the peritoneal cavity can be successfully approached via minimally invasive, local therapies, such as combination therapies using the PICs of the present invention. Thus, PICs of the present invention are therapeutically beneficial.

We claim:

1. A photosensitizer immunoconjugate composition comprising an antibody, a PEGylated polyglutamate chain and at least one photosensitizer molecule, wherein the PEGylated polyglutamate chain is attached to:
a) a non-antigen binding region of the antibody; and
b) at least one photosensitizer molecule such that the photosensitizer molecule is indirectly linked to the antibody through the PEGylated polyglutamate chain.

2. The composition of claim 1 wherein the antibody is selected from the group consisting of whole native antibodies, bispecific antibodies, chimeric antibodies, fusion polypeptides, polyclonal antibodies, monoclonal antibodies, and humanized, monoclonal antibodies.

3. The composition of claim 1, wherein the antibody is tumor-specific.

4. The composition of claim 3, wherein the tumor-specific antibody binds to an epitope on tumors derived from tissues selected from the group consisting of breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

5. The composition of claim 3, wherein the tumor-specific antibody is selected from the group consisting of IMC-C225, EMD 72000, BIWA 1, trastuzumab, rituximab, tositumomab, 2C3, rhuMAb VEGF, sc-321, AF349, BAF349, AF743, BAF743, MAB 743, AB1875, Anti-Flt-4AB3127, FLT41-A, CAMPATH 1H, 2G7, alpha IR-3, ABX-EGF, MDX-447, SR1, Yb5.B8, 17F.11, anti-p75 IL-2R and anti-p64 IL-2R.

6. The composition of claim 1, wherein the antibody is a tumoricidial antibody.

7. The composition of claim 6, wherein the tumoricidial antibody is selected from the group consisting of IMC-C225, EMD 72000, OvaRex Mab B43.13, anti-ganglioside G(D2) antibody ch14.18, CO17-1A, trastuzumab, rhuMAb VEGF, sc-321, AF349, BAF349, AF743, BAF743, MAB743, AB1875, Anti-Flt-4AB3127, FLT41-A, rituximab, 2C3, CAMPATH 1H, 2G7, Alpha IR-3, ABX-EGF, MDX-447, anti-p75 IL-2R, anti-p64 IL-2R, and 2A11.

8. The composition of claim 1, wherein the immunoconjugate comprises up to aboutloo photosensitizer molecules.

9. The composition of claim 1 wherein the photosensitizer molecule is selected from the group consisting of porphyrins, hydroporphyrins, benzoporphyrins, chlorines, bacteriochlorins, purpurins, porphycenes, verdins, phorbides, pheophorbides, texaphyrins, cyanines, photoactive dyes, and combinations thereof.

10. The composition of claim 9, wherein the photoactive dye is selected from the group consisting of cyanines, methylene blue, rose bengal and fluorescein.

11. The composition of claim 9, wherein the porphyrin is benzoporphyrin monoacid derivative.

12. A method of detecting a target cell in a subject comprising the steps of:
a) localizing a photosensitizer immunoconjugate composition comprising an antibody indirectly linked to a photosensitizer by a PEGylated polyglutamate chain to the target cell;
b) light activating the composition to illuminate the target cell; and
c) detecting the target cell.

13. The composition of claim 12, wherein the antibody is selected from the group consisting of whole native antibodies, bispecific antibodies, chimeric antibodies, fusion polypeptides, polyclonal antibodies, monoclonal antibodies, and humanized, monoclonal antibodies.

14. The composition of claim 12, wherein the antibody is tumor-specific.

15. The composition of claim 14, wherein the tumor-specific antibody binds to an epitope on tumors derived from tissues selected from the group consisting of breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

16. The composition of claim 14, wherein the tumor-specific antibody is selected from the group consisting of IMC-C225, EMD 72000, BIWA 1, trastuzumab, rituximab, tositumomab, 2C3, rhuMAb VEGF, sc-321, AF349, BAF349, AF743, BAF743, MAB 743, AB1875, Anti-Flt-4AB3127, FLT41-A, CAMPATH 1H, 2G7, alpha IR-3, ABX-EGF, MDX-447, SR1, Yb5.B8, 17F.11, anti-p75 IL-2R and anti-p64 IL-2R.

17. The composition of claim 12, wherein the antibody is a tumoricidial antibody.

18. The composition of claim 17, wherein the tumoricidial antibody is selected from the group consisting of IMC-C225, EMD 72000, OvaRex Mab B43.13, anti-ganglioside G(D2) antibody ch14.18, CO17-1A, trastuzumab, rhuMAb VEGF, sc-321, AF349, BAF349, AF743, BAF743, MAB743, AB1875, Anti-Flt-4AB3127, FLT41-A, rituximab, 2C3, CAMPATH 1H, 2G7, Alpha IR-3, ABX-EGF, MDX-447, anti-p75 IL-2R, anti-p64 IL-2R, and 2A11.

19. The composition of claim 12, wherein the immunoconjugate comprises up to about 100 photosensitizer molecules.

20. The composition of claim 12, wherein the photosensitizer molecule is selected from the group consisting of porphyrins, hydroporphyrins, benzoporphyrins, chlorines, bacteriochlorins, purpurins, porphycenes, verdins, phorbides, pheophorbides, texaphyrins, cyanines, photoactive dyes, and combinations thereof.

21. The composition of claim 20, wherein the photoactive dye is selected from the group consisting of cyanines, methylene blue, rose Bengal and fluorescein.

22. The composition of claim 20, wherein the porphyrin is benzoporphyrin monoacid derivative.

23. The method of claim 12 wherein the tumor cell growth and/or proliferation comprises a neoplastic disease selected from the group consisting of melanoma, neuroblastoma, glioma, sarcoma, lymphoma, ovarian, prostate, colorectal and small cell lung cancers.

24. A method of reducing tumor cell growth and/or proliferation in a subject comprising the steps of:
a) providing a therapeutically effective amount of a photosensitizer immunoconjugate composition comprising an antibody indirectly linked to photosensitizer by a PEGylated polyglutamate chain to the tumor cell, wherein the antibody binds with specificity to an epitope present on the surface of a tumor cell;
b) light-activating the composition to produce phototoxic species; and
c) inhibiting the tumor cell growth and/or proliferation.

25. The composition of claim 24, wherein the antibody is selected from the group consisting of whole native antibodies, bispecific antibodies, chimeric antibodies, fusion polypeptides, polyclonal antibodies, monoclonal antibodies, and humanized, monoclonal antibodies.

26. The composition of claim 24, wherein the antibody is tumor-specific.

27. The composition of claim 26, wherein the tumor-specific antibody binds to an epitope on tumors derived from tissues selected from the group consisting of breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

28. The composition of claim 26, wherein the tumor-specific antibody is selected from the group comprising IMC-C225, EMD 72000, BIWA 1, trastuzumab, rituximab, tositumomab, 2C3, rhuMAb VEGF, sc-321, AF349, BAF349, AF743, BAF743, MAB 743, AB1875, Anti-Flt-4AB3127, FLT41-A, CAMPATH 1H, 2G7, alpha IR-3, ABX-EGF, MDX-447, SR1, Yb5.B8, 17F.11, anti-p75 IL-2R and anti-p64 IL-2R.

29. The composition of claim 24, wherein the antibody is a tumoricidial antibody.

30. The composition of claim 29, wherein the tumoricidial antibody is selected from the group consisting of IMC-C225, EMD 72000, OvaRex Mab B43.13, anti-ganglioside G(D2) antibody ch14.18, CO17-1A, trastuzumab, rhuMAb VEGF, sc-321, AF349, BAF349, AF743, BAF743, MAB743, AB1875, Anti-Flt-4AB3127, FLT41-A, rituximab, 2C3, CAMPATH 1H, 2G7, Alpha IR-3, ABX-EGF, MDX-447, anti-p75 IL-2R, anti-p64 IL-2R, and 2A11.

31. The composition of claim 24, wherein the immunoconjugate comprises up to about 100 photosensitizer molecules.

32. The composition of claim 24, wherein the photosensitizer molecule is selected from the group consisting of porphyrins, hydroporphyrins, benzoporphyrins, chlorines, bacteriochlorins, purpurins, porphycenes, verdins, phorbides, pheophorbides, texaphyrins, cyanines, photoactive dyes, and combinations thereof.

33. The composition of claim 32, wherein the photoactive dye is selected from the group consisting of cyanines, methylene blue, rose bengal and fluorescein.

34. The composition of claim 32, wherein the porphyrin is benzoporphyrin monoacid derivative.

35. A method of reducing tumor cell growth and/or proliferation in a subject comprising the steps of:
a) providing a therapeutically effective amount of a photosensitizer immunoconjugate composition comprising a antibody indirectly linked to photosensitizer by a PEGylated polyglutamate chain to the tumor cell, wherein the antibody binds with specificity to an epitope present on the surface of a tumor cell and exerts an inhibitory effect on growth and/or proliferation of the tumor cell;
b) light-activating the composition to produce phototoxic species; and
c) inhibiting the tumor cell growth and/or proliferation.

36. The composition of claim 35, wherein the antibody is selected from the group consisting of whole native antibodies, bispecific antibodies, chimeric antibodies, fusion polypeptides, polyclonal antibodies, monoclonal antibodies, and humanized, monoclonal antibodies.

37. The composition of claim 35, wherein the antibody is tumor-specific.

38. The composition of claim 37, wherein the tumor-specific antibody binds to an epitope on tumors derived from tissues comprising breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

39. The composition of claim 37, wherein the tumor-specific antibody is selected from the group consisting of IMC-C225, EMD 72000, BIWA 1, trastuzumab, rituximab, tositumomab, 2C3, rhuMAb VEGF, sc-321, AF349, BAF349, AF743, BAF743, MAB 743, AB1875, Anti-Flt-4AB3127, FLT41-A, CAMPATH 1H, 2G7, alpha IR-3, ABX-EGF, MDX-447, SR1, Yb5.B8, 17F.11, anti-p75 IL-2R and anti-p64 IL-2R.

40. The composition of claim 35, wherein the antibody is a tumoricidial antibody.

41. The composition of claim 40, wherein the tumoricidial antibody is selected from the group consisting of IMC-C225, EMD 72000, OvaRex Mab B43.13, anti-ganglioside G(D2) antibody ch14.18, CO17-1A, trastuzumab, rhuMAb VEGF, sc-321, AF349, BAP349, AF743, BAF743, MAB743, AB1875, Anti-Flt-4AB3127, FLT41-A, rituximab, 2C3, CAMPATH 1H, 2G7, Alpha IR-3, ABX-EGF, MDX-447, anti-p75 IL-2R, anti-p64 IL-2R, and 2A11.

42. The composition of claim 35, wherein the immunoconjugate comprises up to about 100 photosensitizer molecules.

43. The composition of claim 35, wherein the photosensitizer molecule is selected from the group consisting of porphyrins, hydroporphyrins, benzoporphyrins, chlorines, bacteriochlorins, purpurins, porphycenes, verdins, phorbides, pheophorbides, texaphyrins, cyanines, photoactive dyes, and combinations thereof.

44. The composition of claim 43, wherein the photoactive dye is selected from the group consisting of cyanines, methylene blue, rose bengal and fluorescein.

45. The composition of claim 43, wherein the porphyrin is benzoporphyrin monoacid derivative.

46. The method of claim 35, wherein the tumor cell growth and/or proliferation comprises aneoplastic disease selected from the group consisting of melanoma, neuroblastoma, glioma, sarcoma, lymphoma, ovarian, prostate, colorectal and small cell lung cancers.

47. A method of reducing tumor cell growth and/or proliferation in a subject comprising the steps of:
a) providing a therapeutically effective amount of an indirectly linked photosensitizer immunoconjugate composition comprising an antibody indirectly linked to a photosensitizer by a PEGylated polyglutamate chain to the tumor cell, wherein the antibody binds with specificity to a first epitope present on the surface of a tumor cell;
b) providing a therapeutically effective amount of a second antibody to the tumor cell, wherein the antibody binds with specificity to a second epitope present on the surface of a tumor cell and exerts an inhibitory effect on growth and/or proliferation of the tumor cell;
c) light-activating the tumor cell to produce phototoxic species; and
d) inhibiting growth and/or proliferation of the tumor cell.

48. The composition of claim 47, wherein the antibody is selected from the group consisting of whole native antibodies, bispecific antibodies, chimeric antibodies, fusion polypeptides, polyclonal antibodies, monoclonal antibodies, and humanized, monoclonal antibodies.

49. The composition of claim 47, wherein the antibody is tumor-specific.

50. The composition of claim 49, wherein the tumor-specific antibody binds to an epitope on tumors derived from tissues selected from the group consisting of breast, prostate, colon, lung, pharynx, thyroid; lymphoid, lymphatic, larynx, esophagus, oral mucosa, bladder, stomach, intestine, liver, pancreas, ovary, uterus, cervix, testes, dermis, bone, blood and brain.

51. The composition of claim 49, wherein the tumor-specific antibody is selected from the group comprising IMC-C225, EMD 72000, BIWA 1, trastuzumab, rituximab, tositumomab, 2C3, rhuMAb VEGF, sc-321, AF349, BAF349, AF743, BAF743, MAB 743, AB1875, Anti-Flt-4AB3127, FLT41-A, CAMPATH 1H, 2G7, alpha IR-3, ABX-EGF, MDX-447, SR1, Yb5.B8, 17F.11, anti-p75 IL-2R and anti-p64 IL-2R.

52. The composition of claim 47, wherein the antibody is a tumoricidial antibody.

53. The composition of claim 52, wherein the tumoricidial antibody is selected from the group consisting of IMC-C225, EMD 72000, OvaRex Mab B43.13, anti-ganglioside G(D2) antibody ch14.18, CO17-1A, trastuzumab, rhuMAb VEGF, sc-321, AF349, BAF349, AF743, BAF743, MAB743, AB1875, Anti-Flt-4AB3127, FLT41-A, rituximab, 2C3, CAMPATH 1H, 2G7, Alpha IR-3, ABX-EGF, MDX-447, anti-p75 IL-2R, anti-p64 IL-2R, and 2A11.

54. The composition of claim 47, wherein the immunoconjugate comprises up to about 100 photosensitizer molecules.

55. The composition of claim 47, wherein the photosensitizer molecule is selected from the group consisting of porphyrins, hydroporphyrins, benzoporphyrins, chlorines, bacteriochlorins, purpurins, porphycenes, verdins, phorbides, pheophorbides, texaphyrins, cyanines, photoactive dyes, and combinations thereof.

56. The composition of claim 55, wherein the photoactive dye is selected from the group consisting of cyanines, methylene blue, rose Bengal and fluorescein.

57. The composition of claim 55, wherein the porphyrin is benzoporphyrin monoacid derivative.

58. The method of claim 47, wherein the tumor cell growth and/or proliferation comprises a neoplastic disease selected from the group consisting of melanoma, neuroblastoma, glioma, sarcoma, lymphoma, ovarian, prostate, colorectal and small cell lung cancers.

59. A process for preparing an indirectly linked photosensitizer immunoconjugate composition comprising the steps of:
a) preparing a PEGylated polyglutamate chain
b) attaching photosensitizer to a PEGylated polyglutamate chain, and
c) attaching a PEGylated polyglutamate chain to a non-antigen binding region of an antibody
whereby the antibody is indirectly linked to the photosensitizer through the PEGylated polyglutamate chain.

60. A process for preparing an indirectly linked photosensitizer immunoconjugate composition comprising the steps of:
a) preparing a PEGylated polyglutamate chain
b) attaching photo sensitizer to a PEGylated polyglutamate chain
c) activating the photosensitizer-PEG-polyglutamate composition to create a suitable reactive group
d) activating an antibody to create a suitable reactive group, and
e) attaching a PEGylated polyglutamate chain to a non-antigen binding region of the antibody
whereby the antibody is indirectly linked to the photosensitizer through the PEGylated polyglutamate chain.

61. The process of claim 60, wherein the photosensitizer-PEG-polyglutamate composition is activated with hydrazine to form a hydrazide on the carboxylic acid terminus of a glutamate residue, and purified by column chromatography.

62. The process of claim 60, wherein the antibody is activated by oxidation of the hydroxyl groups on the carbohydrates of the hinge region of the antibody, and purified by column chromatography.

63. The process of claim 60, wherein the activated antibody is conjugated to the activated photosensitizer-PEG-polyglutamate composition by forming an amide bond between the oxidized hydroxyl group in the hinge region of the activated antibody and a hydrazide group of the activated photosensitizer-PEG-polyglutamate.

64. The process of claim 60, wherein the photosensitizer-PEG polyglutamate is linked to a lysine residue in the hinge region of the antibody.
